(19) United States
(12) Patent Application Publication
(10) Pub. No.: US 2010/0137718 A1
(43) Pub. Date: Jun. 3, 2010

(54) BIDIMENSIONAL ULTRASONIC ARRAY FOR VOLUMETRIC IMAGING

(76) Inventors: Massimo Pappalardo, Roma (IT); Giosue Caliano, Roma (IT); Alessandro Caronti, Roma (IT); Alessandro Stuart Savoia, Roma (IT); Philipp Gatta, Roma (IT); Cristina Longo, Pietramontecorvino (IT); Vito Bavaro, Bitritto (IT)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/522,734

(22) PCT Filed: Nov. 23, 2007
(86) PCT No.: PCT/EP2007/062773

§ 371 Date: Jul. 10, 2009

(30) Foreign Application Priority Data
Jan. 12, 2007 (EP) 07100503.7

(51) Int. Cl. A61B 8/14

(52) U.S. Cl. 600/459

(57) ABSTRACT

Ultrasound transducer comprising an array of electro-acoustic micro-cells, a first and a second group of transducer elements arranged substantially along two directions (x, y), each element being defined by a group of micro-cells of the array, at least part of the micro-cells of each group being electrically interconnected by a first connection pattern having shape with main orientation along one of the two directions (x, y), characterized in that each group of micro-cells defining each element comprises micro-cells interconnected by further connection pattern or patterns having shape with main orientation along the other of the two directions (y, x).

### CROSS REFERENCES TO RELATED APPLICATIONS

This application is a National Stage filing of International Application PCT/EP2007/062773, filed Nov. 23, 2007, claiming priority to European Application No. EP 07100503.7, filed Jan. 12, 2007, entitled “BIDIMENSIONAL ULTRASONIC ARRAY FOR VOLUMETRIC IMAGING”. The subject application claims priority to PCT/EP2007/062773 and to European Application No. EP 07100503.7 and both references are expressly incorporated by reference herein, in their entirety.

### BACKGROUND OF THE INVENTION

The invention relates to a bidimensional transducer array for ultrasonic imaging.

In the last years an increasing interest in ultrasonic systems capable of generating ultrasound volumetric images has been shown. These systems aim at performing three-dimensional ultrasound imaging by acquiring a succession of section planes of the body under examination, processing the individual reflected echoes in each plane and constructing a three-dimensional image memory which associates the received echo signals to a set of voxels, i.e. image dots of a three-dimensional image, while accounting for the relative position of the individual scan planes. During image acquisition, the whole object volume is generally scanned, through successive two-dimensional section planes staggered to a predetermined extent to cover the whole object volume. Then, the signals of the reflected echoes are processed and transformed into image data in the form of a three-dimensional matrix of image dots. This process must be substantially exhaustive to allow the desired image to be displayed in a plane having any spatial orientation and crossing the object volume. The more intuitive way of achieving a volumetric scanning is by moving a mono-dimensional probe to cover the entire object volume. Such volumetric scanning can be obtained by manually oscillating the entire probe, or in an improved manner, such as the one disclosed in U.S. Pat. No. 6,572,548, by means of a so called 3D motorized probe in which the transducer array can be oscillated by a stepped electric motor around an axis of rotation which is oriented parallel or which is coincident with the longitudinal central axis of the array of transducers.

Volumetric images of better quality can be obtained through bidimensional arrays. In this type of transducers, an ultrasonic beam is swept over a region of interest by electronic means which electronically generate time delays for acoustic radiation from each element of the transducer. Thanks to this technique, the ultrasonic beam, which is generated by the acoustic contributions from all the elements, may be focused on one point, line or area of the region of interest, or steered.

However, this type of two-dimensional transducer array has the drawback of requiring a relatively large number of elements to obtain a sufficient resolution, whereby the cable that connects the elements to the controller shall have a large number of conductors, particularly at least one conductor per element.

Two different arrangements are known in the art to obviate this drawback. A first known arrangement provides the use of a multiplexer and a cable having as many conductors as the elements of a subset, whose conductors are alternately switched on different elements by the multiplexer. In addition to cost problems, the multiplexer is still a space-requiring electronic device, therefore the problem is only partly solved. Also, while the multiplexing process allows to use cables having a reduced number of conductors, i.e. smaller cables, it requires longer times, as the whole transducer array is excited with a transducer subset exciting sequence, whereby a longer beam forming time shall be considered, in addition to focusing or steering delays.

An alternative arrangement is known as a sparse array transducer. Sparse arrays are two-dimensional arrays in which not all elements are connected to the controller or not all the elements are present. Hence, the number of conductors in the cable for connecting the transducer to the control apparatus is actually reduced, but the acoustic signal dynamic range i.e. the major to minor lobe ratio is also reduced. Secondary lobes are in fact related to the number of elements in the array.

A few examples of the foregoing arrangements are further detailed in U.S. Pat. No. 5,537,367 and U.S. Pat. No. 6,419,633.

In the prior art, there are no transducer bidimensional arrays that provide a good dynamic range, i.e. a sufficient major to minor lobe ratio, a good resolution, and a relatively low cost, allowing them to be used with low-price ultrasonic imaging apparatus.

From the technical point of view, the solution to this problem requires two contrasting requirements to be fulfilled. In fact, the attainment of high resolution and dynamic range, i.e. an optimized major to minor or side lobe ratio requires the provision of a large number of elements, whereas transducer size and cost reduction requires a reduction of the number of elements, which affects resolution and dynamic range.

Therefore, the need arises of providing ultrasonic transducer arrays, which have a small size and a sufficient number of elements, such as to provide an optimized resolution and an optimized dynamic range, and which can be fabricated at such costs as to be able to be used with low-price ultrasonic imaging apparatus, i.e. with a limited number of channels.

The invention achieves the above purposes by providing a bidimensional transducer array of the electrostatic type in a particularly advantageous configuration.

Electrostatic ultrasonic transducers, made of a thin metallized membranes (mylar) typically stretched over a metallic plate, known as “backplate”, have been used since 1950 for emitting ultrasounds in air, while the first attempts of emission in water with devices of this kind were on 1972. These devices are based on the electrostatic attraction exerted on the membrane which is forced to flexurally vibrate when an alternate voltage is applied between it and the backplate; during reception, when the membrane is set in vibration by an acoustic wave, incident on it, the capacity modulation due to the membrane movement is used to detect the wave.

More specifically, with reference to FIG. 1, the electrostatic transducer 1, the most known application of which is the condenser microphone, is made of a membrane 2 stretched by a tensile radial force τ in front of a backplate 3, through a suitable support 4 which assures a separation distance d_{g} between membrane 2 and backplate 3.

If the membrane 2 is provided with a metallization 5 and the backplate 3 is conductive, this structure operates as a capacitor of capacitance

$$C = ɛ \cdot \frac{A}{{d}_{g}} \qquad ( 1 )$$

having a fixed electrode (the backplate 3) and a movable one (the membrane 2) both of area A, being ε the dielectric constant of air. By applying a continuous voltage V_{DC} between the two electrode, through a resistor R, an electric charge Q=V_{DC} C distributes along them. An incident acoustic wave puts in flexural vibration the membrane 2 and the related deformation makes the distance d_{g} between the fixed electrode and the movable one vary, and thus consequently the capacitance C of the structure. The variation of capacitance, for the same charge Q, is balanced by an opposite variation of voltage and thus, as a result, at the ends of terminal M3, separated from the movable electrode through the blocking capacitor C_{b}, there appears an alternate voltage V of frequency equal to the one of the incident acoustic wave and of amplitude proportional, through surface A of the membrane 2, to the amplitude of the incident pressure. Such alternate voltage V may be detected on the resistor Rᵢₙ when terminal M3 is connected to terminal M2 through switch 6.

In order to generate acoustic waves in a fluid, an alternate voltage V_{AC} is superimposed to the continuous voltage V_{DC}, by connecting terminal M3 to terminal M1 (as shown in FIG. 1). Because of the electrostatic attraction force

$$F = ɛ \frac{ A \cdot {( {V}_{D C} + {V}_{A C} )}^{2}}{2 {d}_{g}} \qquad ( 2 )$$

the membrane 2 is forced to flexurally oscillate with a vibration amplitude proportional to the applied alternate voltage V_{AC}. The correct equations putting the electric parameters, voltage and current, in relation with the mechanical ones, vibration velocity and force exerted by the membrane on the fluid, are well known and obtainable in literature.

It has been recently introduced a new generation of electrostatic ultrasonic transducers known as “surface micromachined ultrasonic transducers” or also as capacitive Micromachined Ultrasonic Transducers (cMUTs). The cMUTs, and their related processes of fabrication with the silicon micro-machining technology, have been disclosed, for example, by X. Jin, I. Ladabaum, F. L. Degertekin, S. Calmes, e B. T. Khuri-Yakub in “Fabrication and characterization of surface micromachined capacitive ultrasonic immersion transducers”, J. Microelectromech. Syst., vol. 8(1), pp. 100-114, September 1998, by X. Jin, I. Ladabaum, e B. T. Khuri-Yakub in “The microfabrication of capacitive ultrasonic Transducers”, Journal of Microelectromechanical Systems, vol 7 No 3, pp. 295-302, September 1998, by I. Ladabaum, X. Jin, H. T. Soh, A. Atalar and B. T. Khuri-Yakub in “Surface micromachined capacitive ultrasonic transducers”, IEEE Trans. Ultrason. Ferroelect. Freq. Contr., vol. 45, pp. 678-690, May 1998, in the U.S. Pat. No. 5,870,351 by I. Ladabaum et al., in the U.S. Pat. No. 5,894,452 by I. Ladabaum et al., and by R. A. Noble, R. J. Bozeat, T. J. Robertson, D. R. Billson and D. A. Hutchins in “Novel silicon nitride micromachined wide bandwidth ultrasonic transducers”, IEEE Ultrasonics Symposium isbn: 0-7803-4095-7, 1998.

These transducers are made of arrays of electrostatic micro-cells, electrically interconnected so as to be driven in phase, obtained through surface micromachining.

The fundamental steps of a conventional process for fabricating cMUT transducer micro-cells through silicon micro-machining technology are described in U.S. Pat. No. 5,894,452, and they are shown in FIG. 2.

As shown in FIG. 2a, a sacrificial film 12 (for example silicon dioxide), the thickness H of which will define the distance d_{g} between micro-plate 9 and the backplate, is deposited on a silicon substrate 11.

FIG. 2b shows that a second structural film 13, for example of silicon nitride, of thickness h′, is deposited on the first sacrificial film 12; a narrow hole 14 (etching via) is formed in it, through classical photolithographic techniques, in order to create a path, shown in FIG. 5c, for removing the underlying sacrificial film 12.

A selective liquid solution is used for etching only the sacrificial film 12, whereby, as shown in FIG. 2d, a large cavity 15, circular in shape and having radius dependent on the etching time, is created under the structural film 13 which remains suspended over the cavity 15 and which is the micro-plate 9 of the underlying micro-cell.

Finally, the etching hole 14 is sealed by depositing a second silicon nitride film 16, as shown in FIG. 2e. With reference to FIG. 2f, the cells are completed by evaporating a metallic film 17 on the micro-plate 9 which is one of the electrodes, while the second electrode 18 is made of the silicon substrate 11 heavily doped and hence conductive.

In order to obtain transducers capable to operate in the range 1-15 MHz, typical in many echographic applications for non-destructive tests and medical diagnostics, the micro-membrane lateral size of each cell is of the order of ten microns. Moreover, in order to achieve large bandwidths and the required sensitivity, each element of a cMut transducer is typically formed by some thousands of micro-cells connected together by using appropriate metallization patterns.

The applicant has now observed that by acting on the form and/or shape of the metallization patterns, it is possible to combine elementary micro-cells to form transducer elements of arbitrary geometry. This can be used for solving the problem posed above, i.e. how to provide ultrasonic transducer arrays, which have a small size and a sufficient number of elements, such as to provide an optimized resolution and an optimized dynamic range, and which can be fabricated at such costs as to be able to be used with low-price ultrasonic imaging apparatus, i.e. with a limited number of channels.

The invention achieves the above purposes by providing an ultrasound transducer comprising an array of electro-acoustic micro-cells, a first and a second group of transducer elements arranged substantially along two directions (x, y), each element being defined by a group of micro-cells of the array, at least part of the micro-cells of each group being electrically interconnected by a first connection pattern having shape with main orientation along one of the two directions (x, y), wherein each group of micro-cells defining each element comprises micro-cells interconnected by further connection pattern or patterns, electrically isolated from the first connection pattern, having shape with main orientation along the other of the two directions (y, x). In practical the micro-cells are connected to form elements which are geometrically superimposed along two main directions, preferably, but not exclusively, orthogonal. The orientation of each element is defined by the first connection pattern, which substantially extends along the entire length of the element.

The overlay between the elements can be obtained by using geometries that allow to compenetrate groups of micro-cells so that the resulting bidimensional array is the result of two independent mono-dimensional arrays differently orientated with elements superimposed. The same effect of overlay of elements can also be obtained by properly connecting both the electrodes of the micro-cells so that the same micro-cells can be shared between different elements.

If n is the number of elements of the first mono-dimensional transducer and m the number of elements of the second mono-dimensional transducer, the resulting bidimensional array contains m+n elements, while a corresponding full bidimensional array of m rows and n columns contains m×n elements, thus achieving a reduction in the number of elements by a factor k=n×m/(n+m). That means that in case of a full array of 64×64=4096 elements, the transducer according to the present invention is formed only by 128 elements with evident simplification in terms of wiring and driving electronic circuits.

Preferably the elements of the first group and the elements of the second group are arranged to form two mono-dimensional arrays, at least partially overlapped, respectively oriented along the x and y direction. The metallization patterns are generally provided in at least two layers of the transducer. According to an embodiment, the metallization patterns on the first layer have the form of curved and/or polygonal lines substantially defining the direction of the elements of the first group (x) and the metallization patterns on the second layer have the form of curved and/or polygonal lines substantially defining the direction of the elements of the second group (y). The layers are isolated thus allowing the connecting lines on different layers to be crossed. The number of lines on the first layer define the number of elements of the first group and the number of lines on the second layer define the number of elements of the second group.

Advantageously, the micro-cells have first and second electrode, each element comprising a group of micro-cells having the first electrodes connected together, the connection pattern of such first electrodes defining a path substantially along the direction of the element. Typically the micro-cells forming the elements of the first group are connected on a first layer, while the micro-cells forming the elements of the second group are connected on a second layer.

According to an embodiment, the second electrode of the micro-cells of the array is commonly connected and each element of a group is formed by micro-cells of one group having first electrodes connected together to define a path substantially along the direction of the element and by interleaved micro-cells of other subgroups, the first electrodes of said micro-cells of the other groups being so connected to define a path substantially along the direction of the elements of the other group. Each element of the first group typically comprises intermingled subgroups of micro-cells belonging to elements of the second group. Particularly each element of the first group is defined by subgroups of micro-cells connected together, alternated by subgroups of micro-cells connected to form elements of the second group in a chess-board-like disposition.

According to an embodiment each element of a group is formed by an elementary matrix of interconnected micro-cells having an arbitrary shape repeated along the element to fill a rectangular area of the array, the long axis of the rectangle defining the direction of the element. Particularly the elementary matrixes of the area are connected together to define a pattern substantially along the direction of the element, the remaining elementary matrixes of the element being partially connected together according to patterns substantially parallel to the short axis of the rectangle. The patterns substantially parallel to the short axis of the rectangle can be oriented along directions parallel to the long axis of the rectangles forming the elements of the other group.

Preferably, the elementary matrixes have the shape of quadrilaterals, e.g. squares or rhombuses, interconnected through the vertexes so that two consecutive quadrilaterals are connected only on one vertex of each quadrilateral. One side of the quadrilaterals may be parallel to one of the axis of the rectangle. Alternatively all the sides of the quadrilaterals may be oblique with respect to the axes of the rectangle, the connection path between two vertexes being parallel to one of the axis of the rectangle.

Assuming for example a matrix arrangement of the micro-cells, it is possible to obtain each element of a first mono-dimensional array placed along the x axis (elements of the first type) by alternatively connecting subgroups of micro-cells with vertical connecting lines having typically a zigzag behavior. Each element of a second mono-dimensional array placed along the y axis (elements of the second type) is correspondingly formed by alternatively connecting subgroups of micro-cells with horizontal connecting lines having typically a zigzag behavior in the resulting interlaced matrix arrangement of FIG. 4.

According to an embodiment, each element of a group comprises subgroups of micro-cells having the second electrodes connected together, the connection pattern of such second electrodes defining a path substantially along the direction of the elements of the other group. Each element of a group can thus share at least part of the micro-cells of elements of the other group. In this configuration there's no need to spatially differentiate the micro-cells belonging to different elements as the short circuit between the cells is avoided by using both the electrodes of each micro-cell. Preferably, each element is rectangularly shaped, the elements of the two groups being orthogonal, each element of a group being formed by micro-cells also belonging to elements of the other group.

Advantageously, any element of a group is formed by micro-cells having first or second electrodes interconnected by at least a metallization pattern substantially extending along the entire surface of the element, the micro-cells forming an element of the first group having the first electrodes interconnected and the micro-cells forming an element of the second group having the second electrodes interconnected or vice versa. Each element of a group is thus defined by the metallization pattern interconnecting the first electrodes and each element of the other group is defined by the metallization pattern interconnecting the second electrodes or vice versa.

The micro-cells can also be connected by external connecting means like switches, but they are preferably connected through metallization patterns provided in the array, in particular in at least two layers of the transducer preferably deposited during the microfabrication process.

Advantageously the micro-cells are connected to define a bidimensional array of cross elements arranged in rows and columns, such as, for example, 128 rows and 64 columns, provided with connecting pads to allow the connection of each row and each column of the array to electronic driving means.

The transducer according to the invention is typically plane. However it can be advantageously also of the convex type, i.e. the micro-cells are placed on a curved surface, thus allowing a widening of the field of view of the transducer as it is explained in K. A. Wong, S. Panda and I. Ladabaum, “Curved Micromachined Ultrasonic Transducers”, 2003 IEEE Ultrasonics Symposium.

According to another aspect, the invention relates to a combination of the transducer and an electronic circuit particularly adapted for driving such a transducer. The circuit advantageously has driving means for independently driving all the elements of the transducer although just a subset of them may suffice. Due to the fact that the cMUT are microfabricated on silicon, the electronic circuit and the transducer can be integrated on the same chip thus resulting in a particular compact device. However, such a circuit can also be advantageously part of the front-end of an ultrasonic imaging device.

According to an embodiment, the electronic circuit comprises a first and a second beam-former while the transducer is of the matrix type array having m rows and n columns. The rows of the transducer are connected or connectible to said first beam-former, while the columns of the transducer are connected or connectible to said second beamformer. The first and second beam-formers typically comprise amplifying and/or delay and/or summing elements to achieve an independent focusing of the beams generated/received by the columns and the rows of the array. Beam-formers are well-known circuits for those skilled in the art. Beam-formers essentially consist of summing and delay elements for delaying the signal received/transmitted from/to a transducer to allow a focusing of the resulting beam. See for example Dan E. Dudgeon, “Fundamentals of Digital Array Processing,” Proceedings of the IEEE, volume 65, pp. 898-904, June 1977 and B. D. Steinberg, “Digital Beam-forming in Ultrasound,” IEEE Transactions on Ultrasonics, Ferroelectrics, and Frequency Control, volume 39, pp. 716-721, November 1992.

The two beam-formers are advantageously provided for allowing the focusing of the beam received from the transducer. The first beam-former has an input for the connection with the rows and an output for providing a first focused signal while the second beam-former has an input for the connection with the columns of the transducer and an output for providing a second focused signal.

As it can be seen in FIG. 10, if the outputs of the two beam-formers are connected together through a linear circuit, like for example an adder, the resulting beam pattern is not optimized due to the presence of side lobes. They are mainly due to the main lobes of the two arrays on the non-superimposed focused area, as it is schematically depicted in FIG. 10d and explained in detail below. For this reason the outputs are preferably combined through a non-linear circuit that can be for example a multiplier and/or a logarithmic and/or a cross-correlation circuit. In fact, if the two outputs are not added, but for example multiplied, the non-superimposed area is attenuated thus resulting in a beam pattern with less lateral lobes.

According to an embodiment, the array according to the invention is used in combination with a particularly advantageous beam-forming technique based on two consecutive pulse firings. This allows to achieve an optimized beam formation with a transducer having superimposed elements (x, y) as in the present invention. The elements are driven to transmit a first and second pulse causing a target to generate a first and second echo signal. The phase of the pulse transmitted by the x and y elements are advantageously changed, in particular reversed, during the second transmission so that a sidelobe reduction of the resulting beam can be obtained when the echo signals are combined. The method can be further improved by combining the echo signals after the envelope has been extracted.

The method is based on the idea of generating an optimized acoustic field distribution by exploiting the physical phenomenon of phase cancellation, which allows to cancel or at least reduce the undesired components responsible of sidelobe formation. Advantageously the steps of the method comprise:
setting the focusing parameters of each of the two beam-formers;
generating a first transmitting pulse;
feeding each of the two beam-formers with such pulse;
receiving the echo;
achieving an independent beam-forming in reception with each of the two beam-formers;
adding the resulting focused signals to obtain a signal S₁(t);
generating a second transmitting pulse;
feeding one of the two beam-formers with such pulse;
feeding the other of the two beam-formers with the same pulse 180°-phase-shifted;
receiving the echo;
achieving an independent beam-forming in reception with each of the two beam-formers;
adding the resulting focused signals after a 180°-phase shift to obtain a signal S₂(t);
subtracting the S₁(t) and S₂(t) signals to obtain the signal S(t);
extracting the envelope of signal S(t) to be used for imaging purpose.

According to another embodiment, the last two steps are modified in that the signal S(t) to be used for imaging purpose is obtained by subtracting the envelope of the S₁(t) and S₂(t) signals.

According to another aspect the invention relates to an ultrasound imaging apparatus comprising a front-end having a number of channels comprising transmitting and receiving means for driving ultrasonic transducers, specially adapted for being interfaced with a transducer according to the invention. Particularly the apparatus has at least two beam-formers and control means, typically of the programmable type, for controlling the beam formation. Such means is preferably configured to perform the method steps seen above.

Such an ultrasound imaging apparatus is particularly advantageous if provided in combination with a transducer according to the invention, however the skilled person would appreciate that it can also be advantageously used with any kind of transducer arrays, especially when there's the need to optimize the beam formation with a reduced number of echographic channels.

Further improvements of the invention will form the subject of the dependent claims.

The characteristics of the invention and the advantages derived therefrom will be more apparent from the following description of a non-limiting embodiment, illustrated in the annexed drawings.

### BRIEF SUMMARY

Ultrasound transducer comprising an array of electro-acoustic micro-cells, a first and a second group of transducer elements arranged substantially along two directions (x, y), each element being defined by a group of micro-cells of the array, at least part of the micro-cells of each group being electrically interconnected by a first connection pattern having shape with main orientation along one of the two directions (x, y), characterized in that each group of micro-cells defining each element comprises micro-cells interconnected by further connection pattern or patterns having shape with main orientation along the other of the two directions (y, x).

One object of the present disclosure is to describe an improved ultrasound transducer array for ultrasonic imaging.

### BRIEF DESCRIPTION OF THE SEVERAL VIEWS OF THE DRAWINGS

FIG. 1 shows an electrostatic transducer.

FIG. 2 shows a cMUT transducer.

FIG. 3a shows a simplified drawing of the acoustic beam generated by a mono-dimensional linear array with elements oriented along the x axis.

FIG. 3b shows a simplified drawing of the acoustic beam generated by a mono-dimensional linear array with elements oriented along the y axis.

FIG. 3c shows a simplified drawing of the acoustic beam generated by overlaying the acoustic fields generated by the two mono-dimensional linear arrays of FIGS. 3a and 3b.

FIG. 4 shows a highly simplified example (4×4 elements) of a bi-dimensional array (FIG. 4c) obtained by superimposing two mono-dimensional arrays (FIG. 4a and FIG. 4b) having a “chess-board-like” layout.

FIG. 5 shows a highly simplified example (4×4 elements) of a bi-dimensional array (FIG. 5c) obtained by superimposing two mono-dimensional arrays (FIG. 5a and FIG. 5b) having a “rhombus” layout.

FIG. 6 shows a highly simplified example (4×4 elements) of a bi-dimensional array (FIG. 6c) with “floating ground” obtained by connecting the upper electrodes along the y direction and the lower electrodes along the x direction (FIG. 6a and FIG. 6b).

FIG. 7 shows the equivalent electric circuit (only 3+3 elements are shown) of the “floating ground” configuration of FIG. 6 (FIG. 7a) and the related polarization circuit (tee bias) (FIG. 7b).

FIG. 8 shows the equivalent Norton model (FIG. 8a) of the electric circuit of FIG. 7a and the connections with input trans-impedance circuits (FIG. 8b).

FIG. 9 shows a highly simplified diagram of a 4×4 array according to the invention where each row represents an element of a mono-dimensional array oriented along the y direction and each column represents an element of a mono-dimensional array oriented along the x direction, the rows and columns being connected to electronic driving and/or processing circuits.

FIG. 10 schematically shows the pattern of the acoustic beam in reception generated by the two mono-dimensional arrays (FIG. 10a and FIG. 10b) of FIG. 9, the product (FIG. 10c) and the summation (FIG. 10d) of the two patterns.

FIG. 11 shows the directivity diagram on the focal plane obtained with two 32-element mono-dimensional arrays according to the invention respectively by transmitting a pulse in phase and coherently summing the signals so received (FIG. 11a) and by transmitting a pulse 180° out of phase and summing the signals so received after a further 180° phase shift (FIG. 11b).

FIG. 12a shows the directivity diagram on the focal plane obtained by differentiating the envelope of the signals of FIG. 11a and FIG. 11b.

FIG. 12b shows the directivity diagram on the focal plane obtained with a full array of 1024 elements.

FIG. 13 shows a comparison between the images obtained on a spherical phantom with a 64-element array according to the invention (FIG. 13a) and a 1024-element full array (FIG. 13b). The dynamic range of the images, i.e. the ratio in dB of the values corresponding to white and black pixels in the greyscale representation, is 60 dB.

FIG. 14 shows the comparison of the point spread function (psf) obtained with a cross (FIG. 14a) and an interleaved (FIG. 14b) beamforming modality. Also in this example the dynamic range is 60 dB.

FIG. 15 shows the basic steps of a process for fabricating a cMUT transducer according to the invention with PECVD silicon nitride as a membrane structural layer, evaporated chromium as a sacrificial layer, and sputtered aluminium for the metallization.

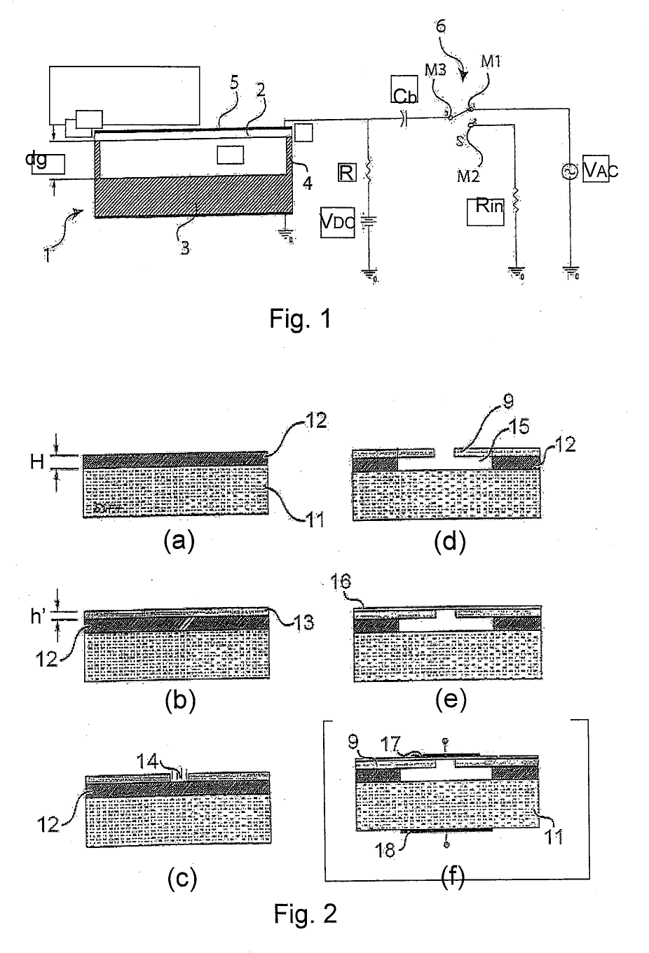

### DETAILED DESCRIPTION

For the purposes of promoting an understanding of the disclosure, reference will now be made to the embodiments illustrated in the drawings and specific language will be used to describe the same. It will nevertheless be understood that no limitation of the scope of the disclosure is thereby intended, such alterations and further modifications in the illustrated device and its use, and such further applications of the principles of the disclosure as illustrated therein being contemplated as would normally occur to one skilled in the art to which the disclosure relates.

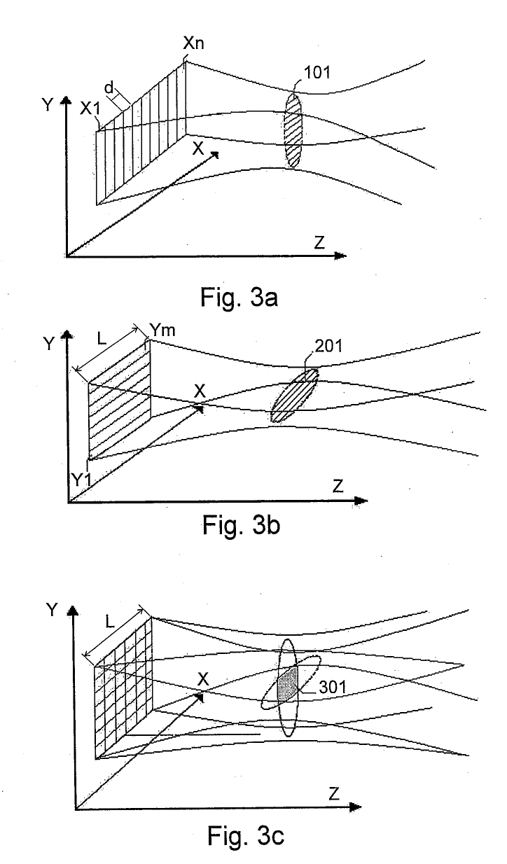

FIG. 3a shows a known linear mono-dimensional transducer having n rectangularly shaped elements (X₁, . . . Xₙ) placed along the x-axis at a distance d between their respective centres. If the beam is not steered, the direction of the acoustic propagation is parallel to the z-axis. By applying appropriate delays to the elements (beamforming), a focused acoustic beam can be obtained like the one schematically depicted in FIG. 3a. Particularly, it is possible to obtain, in the focal plane 101, a very narrow beam with respect to the x-axis direction. The width of the beam in such direction is, at a first approximation, inversely proportional to the number of elements n, to their lateral dimension d and to the working frequency. On the contrary, in the near field, i.e. at distances satisfying the condition z<L2/λ, where λ is the wavelength, the width of the beam with respect to the y-axis is substantially constant and about equal to the height L of the elements.

Similarly, a mono-dimensional array having elements (Y₁, . . . Yₘ) placed along the y-axis, as the one shown in FIG. 3b, will show an acoustic beam in the focal plane 201 narrow with respect to the y-axis direction and approximately equal to L along the x-axis, in the near field.

If the two mono-dimensional arrays (X₁, . . . Xₙ), (Y₁, . . . Yₘ) of FIG. 3a and FIG. 3b can be somehow superimposed in the same region of the space and assuming the transmission medium linear and homogenous, the radiations emitted by the two arrays constructively interfere thus generating a field of pressure whose maximum lies in the shaded zone 301 of the exemplified drawing of FIG. 3c, i.e. in the area shared by the two main lobes of the beams.

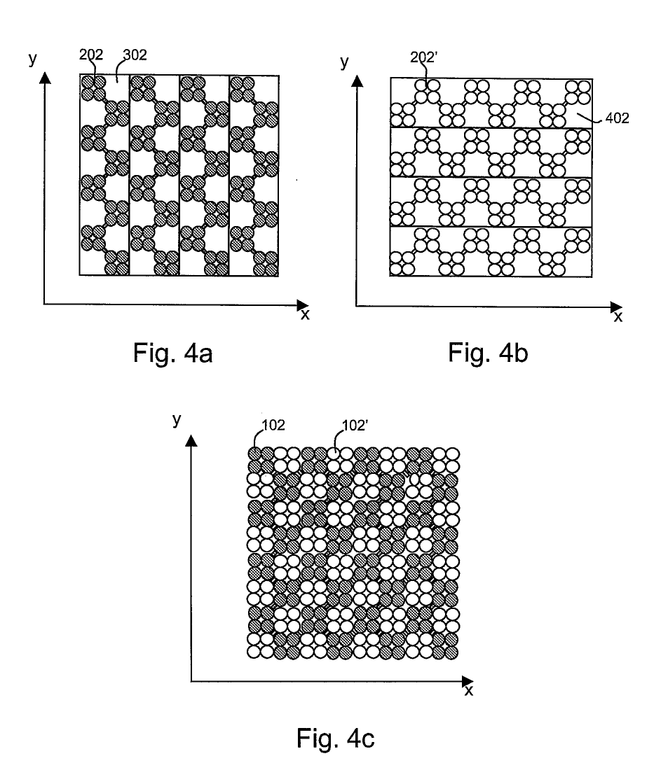

FIGS. 4 to 6 show how such a superimposition can be obtained in three embodiments of a transducer array according to the invention.

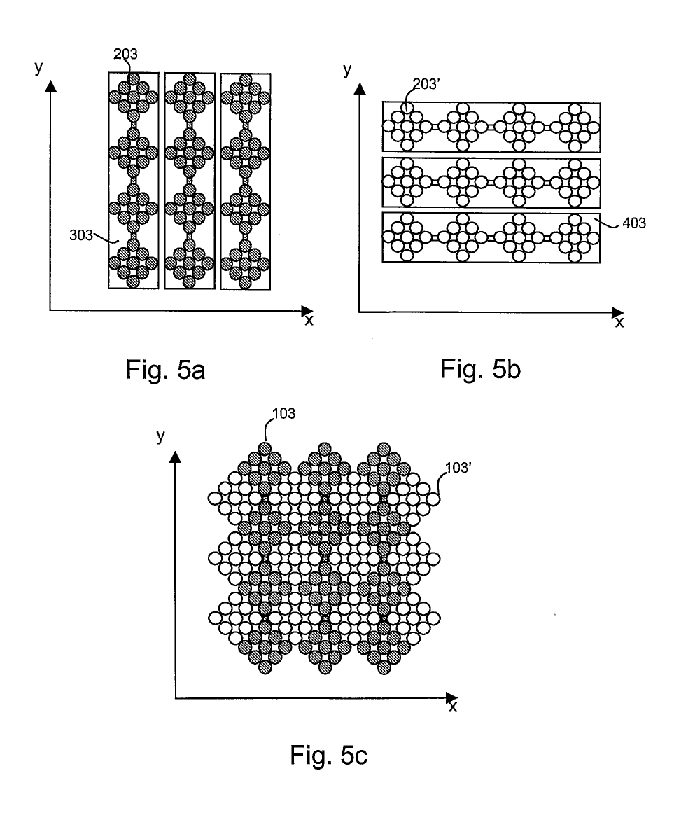

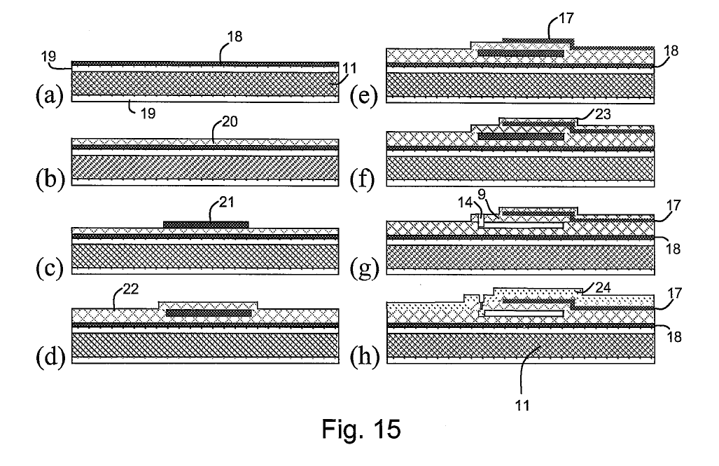

The configurations shown in FIG. 4 and FIG. 5 are obtained by interconnecting through metallization patterns one of the two electrodes of each micro-cells of the array, like electrode 17 of FIG. 2 and FIG. 15. The other electrode 18 is commonly connected, generally to ground, for example through a conductive substrate common to all the micro-cells (see reference 11 of FIG. 2). Being one electrode in common, for avoiding short circuits, the effect of superimposition of the elements is obtained by spatially differentiating the micro-cells in an intermingled layout.

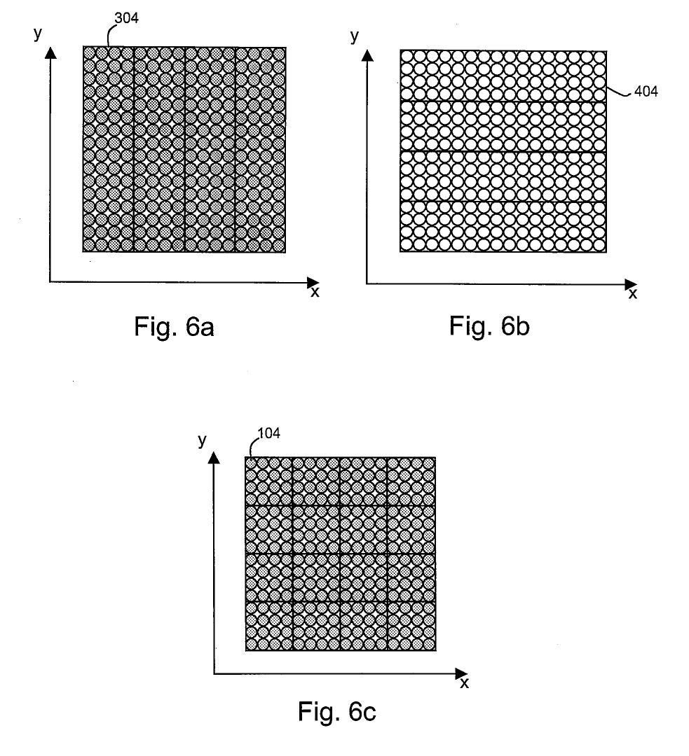

FIG. 6 shows another embodiment of the invention. Here both the electrodes of each micro-cell are used to form the elements of the array. The transducer so obtained is a floating-ground transducer, as no common connection between the micro-cells exists. One process of fabrication of such a transducer is disclosed, for example, in the published international application WO 2006/092820. The transducer disclosed in this document is particularly suitable for being used in ground-floating configurations, although it can also be clearly used in common-ground configurations, like the ones depicted in FIGS. 4 and 5, by connecting together one of the electrodes of each micro-cells through an appropriate metallization layer on the back of the transducer or via the conductive substrate.

Another example of a process of fabrication, which can be used for manufacturing a transducer according to the invention, is disclosed in U.S. Pat. No. 7,074,634. An advantageous improvement of such process is hereinafter disclosed with reference to FIG. 15, which shows the basic fabrication steps of a process using PECVD silicon nitride as a membrane structural layer, evaporated chromium as a sacrificial layer, and sputtered aluminium for the metallization. The device is fabricated onto a silicon wafer 11 covered with thick thermal silicon dioxide 19 on both sides. After aluminium sputtering and bottom electrode 18 patterning, a thin layer 20 of silicon nitride is deposited by rf-PECVD (FIG. 15(a)-(b)). A chromium layer 21, acting as sacrificial layer, is evaporated and patterned into islands to define the cavities under the membranes 9 (FIG. 15(c)). The etching selectivity of chromium against silicon nitride allows a good control over the cell lateral dimensions and gap height. A first silicon nitride membrane layer 22 is deposited at 350° C. using silane, ammonia, and nitrogen diluted in helium as reactant gases (FIG. 15(d)). The tensile stress of the film is controlled by varying the silane to ammonia flow ratio. An aluminium layer 17 is then sputtered on top of the membranes and patterned to define the top electrodes and interconnections between adjacent cells (FIG. 15(e)). After a second silicon nitride deposition 23, the membranes are released by wet etching of the sacrificial layer 21 through the etching holes 14 defined around the perimeter of the membranes 9 (FIG. 15(f)-(g)). Finally, the etching holes 9 are sealed by a third silicon nitride deposition 24 (FIG. 15(h)). By appropriate acting on the form and/or shape of electrodes and interconnections patterns is possible to combine elementary micro-cells to form transducer elements according to the invention.

With reference to FIG. 4, an array of 256 micro-cells having a commonly connected electrode is depicted. The micro-cells are schematically sketched as circles 102, 102′ placed on a square matrix of 16 micro-cells per side. The micro-cells are connected in groups 202, 202′ to form small squares of 4 micro-cells per side. Each group is further connected to form an array of 4×4 orthogonal elements 402, 302 placed along the x and the y-axis. FIGS. 4a and 4b show the connection patterns used for identifying the main orientation of the 4 elements respectively along the y-axis (FIG. 4a) and along the x-axis (FIG. 4b). The connection pattern of each element has a zigzag behavior resulting in a chess-board-like configuration where contiguous groups of micro-cells are alternatively connected to form each element. The resulting configuration is shown in FIG. 4c. If the distance between the micro-cells is less than the wavelength λ, each element 302, 402, although formed by micro-cells 102, 102′ interconnected by different, isolated, connection patterns, will acoustically behave as if it were formed by 4×16 micro-cells connected together. The result is thus an array of 4×4-superimposed elements.

In FIG. 5 a similar configuration is shown. Here the micro-cells 103, 103′ are connected in groups to form small rhombuses or squares of 9 micro-cells per side 203, 203′, rotated by 45° with respect to the configuration of FIG. 4. The connection pattern of each element has a straight behavior with main orientation along the axis of the element resulting in a chess-board-like configuration like the one of FIG. 4, but rotated by 45° as shown in FIG. 5c. Also in this embodiment the elements 303, 403 are spatially arranged in a complementary way to obtain the desired effect of superimposition.

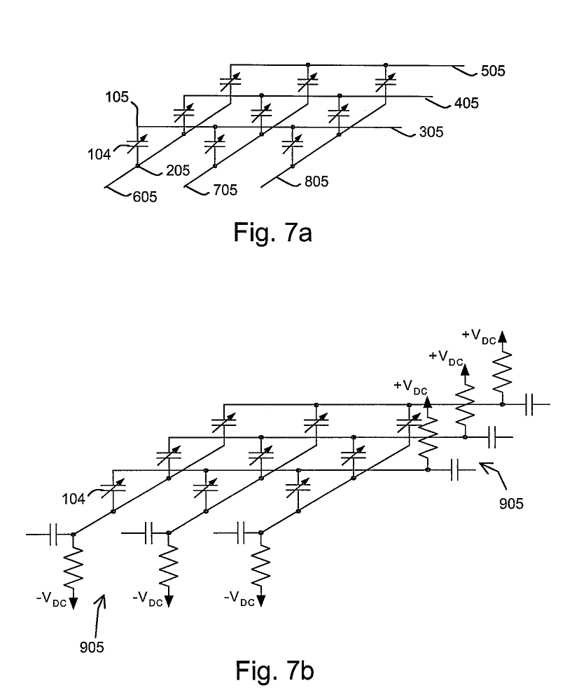

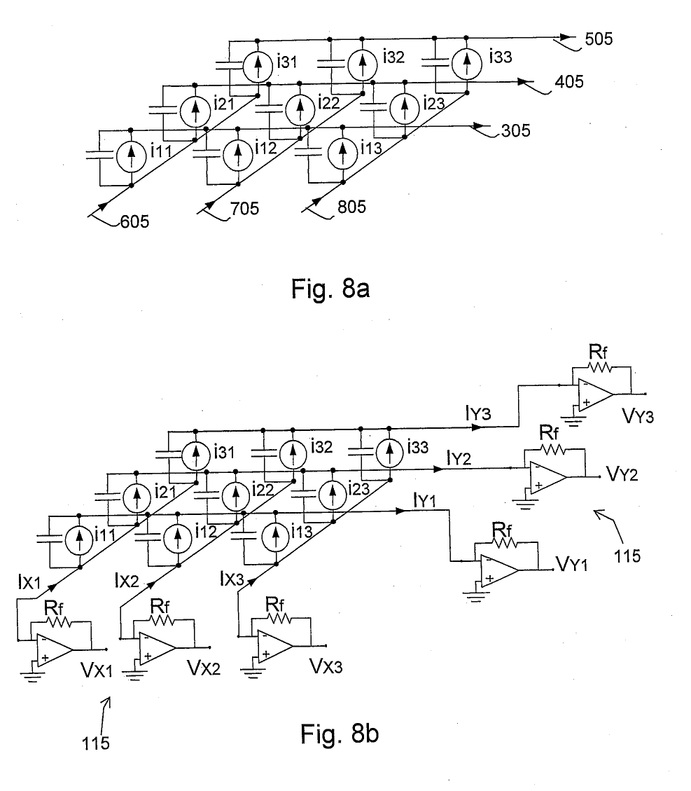

With reference to FIG. 6, an array of 256 micro-cells is depicted. The micro-cells have both electrodes available for connections and are schematically sketched as circles 104 placed on a square matrix of 16 micro-cells per side. Each element 304, 404 shares at least part of the micro-cells of the other element(s). Here there's no need to spatially differentiate the micro-cells belonging to different elements as the short circuit between the cells is avoided by using both the electrodes of each micro-cell 104. Each element 304, 404 is formed by 16×4 micro-cells as shown in FIGS. 6a and 6b with the resulting 4×4 element array of FIG. 6c. The elements 304, 404 are formed by connecting the two electrodes of the micro-cells using different metallization patterns to obtain a configuration of the array similar to those seen above. FIG. 7a illustrates how the connections can be made in a simplified array of 9 micro-cells. Each micro-cell is symbolically represented by a variable concentrated capacitor 104 having top and bottom electrodes 105, 205. By connecting all the top electrodes 105 to a metallization pattern 305, 405, 505 with main orientation along the x direction and all the bottom electrodes 205 to a metallization pattern 605, 705, 805 with main orientation along the y direction, the result is a bi-dimensional array having x and y elements overlaid without common ground electrode. FIG. 7b shows the same configuration of FIG. 7a with a polarization circuit 905 exemplified by a DC voltage applied to each row and column through an RC network. FIG. 8a shows the equivalent Norton model of the electric circuit of FIG. 7a. Each micro-cell is represented by a current generator with impedance in parallel, indicated as with i,j=1 to 3. By driving each row 305, 405, 505 and column 605, 705, 805 of the array is possible to excite all the micro-cells. Particularly the micro-cell iᵢⱼ, i.e. placed at row i and column j, will be excited by a signal which is the difference between the signal applied to element i and element j of the array thus obtaining the desired effect of superimposition of the elements. The same principle can be applied in reception by connecting the elements with input trans-impedance circuits 115 having low input impedance as shown in FIG. 8b. These circuits allow to read the current of each row I_{y1}, I_{y2}, I_{y3} and column Iₓ₁, Iₓ₂, Iₓ₃ of the array, which current is proportional to the acoustic pressure incident on the array averaged on the same rows and columns, thus obtaining the same effect of superimposition of the elements. Such overlay is physical in the sense that different elements share the same micro-cells, while the overlay in the configurations of FIGS. 4 and 5 is acoustic because different elements are formed by different complementary micro-cells, which act as if they were superimposed.

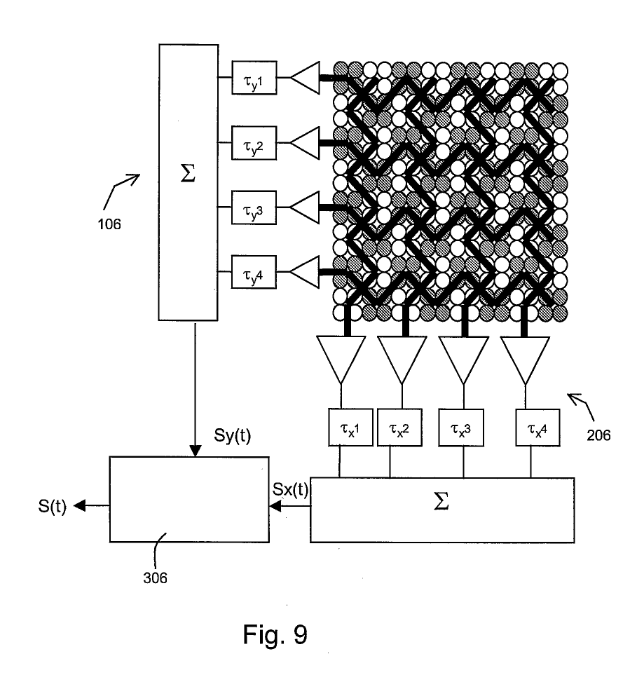

For exploiting the full potentiality of the invention, the transducer elements need to be appropriately interfaced with an electronic circuit, which allows to appropriately manage the acoustic beam formation. The circuit advantageously has focusing means for independently focusing the beam generated/received by each of the two overlaid mono-dimensional arrays of the transducer. With reference to FIG. 9, the electronic circuit comprises a first and a second receiving beamformer 106, 206. The transducer is of the matrix type array having m rows and n columns (m=n=4 in the example of the figure). Beamformers are well-known circuits for those skilled in the art. They essentially consist of summing and delay (τₓ1 . . . τₓ4, τ_{y}1 . . . τ_{y}4) elements for delaying the signal received/transmitted from/to a transducer to allow a focusing of the resulting beam. For further details see the already mentioned articles by Dan E. Dudgeon and B. D. Steinberg.

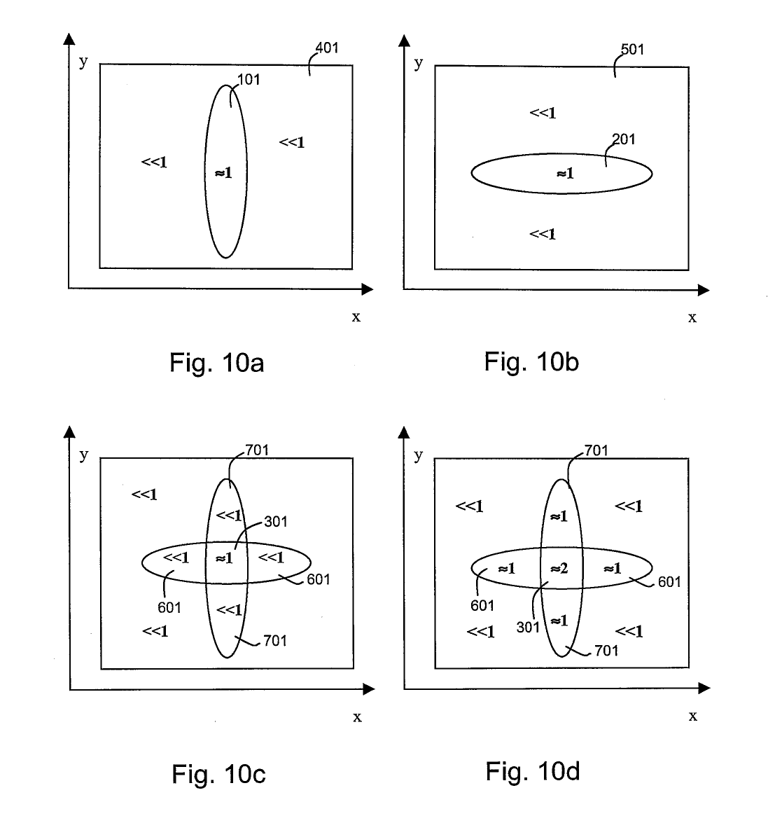

The two beamformers 106, 206 of FIG. 9 are advantageously provided for allowing the focusing of the beam received from the transducer. The first beamformer 106 has an input for the connection with the rows and an output for providing a first focused signal Sy(t) while the second beamformer 206 has an input for the connection with the columns of the transducer and an output for providing a second focused signal Sx(t). FIG. 10a and FIG. 10b respectively schematically show the main lobes 101, 201 of the envelope of the two focused signals Sy(t) and Sx(t). If it is assumed that in the main lobe 101, 201 the signal has amplitude almost “1”, in the region outside the lobe 401, 501 the amplitude is much less than “1” as a result of the focalization (see the symbol <<1 in the figures). This is of course an approximation to render the example clearer. If the outputs of the two beamformers Sx(t) and Sy(t) are combined through a linear circuit 306, like for example an adder, the resulting beam pattern is not optimized due to the presence of side lobes 601, 701 which are mainly due to the main lobes 101, 201 of the two arrays on the non-superimposed focused area, as it is schematically depicted in FIG. 10d where the envelope of the signal S(t)=Sx(t)+Sy(t) is plotted. If the outputs are combined through a non-linear circuit 306 such as a multiplier and/or a logarithmic and/or a cross-correlation circuit, the two outputs are not added and the non-superimposed area is attenuated thus resulting in a beam pattern with less lateral lobes 601, 701 as shown in FIG. 10c. This can be explained with the symbolism so far used. As the multiplication of a signal having amplitude almost “1” with a signal having amplitude much less than “1” is a signal having amplitude much less than “1” in first approximation, if the combination circuit is a multiplier, the resulting focused signal S(t)=Sx(t)·Sy(t) will show a significant amplitude only in the superimposition zone 301, symbolically indicated with ≈1 in FIG. 10c.

Beam formation is a very important issue. Only if side lobes are reduced to acceptable values, the transducer according to the invention may be a valid low-cost alternative to full 2D-arrays. For this reason the applicant has devised a particularly advantageous beamforming technique based on two consecutive pulse firings.

Two-pulse beamforming techniques are known in the field in the so called Harmonic Imaging wherein two consecutive pulses are fired with 180° phase shift and the received signals added to obtain a cancellation of the echoes at the main frequency and thus enhance the second harmonic component for ultrasound contrast imaging. See for example M. F. Bruce, M. A. Averkiou, D. M. Skyba e J. E. Powers, “A generalization of Pulse Inversion Doppler”, 2000 IEEE Ultrasonic Symposium Proc., 1903-1906 and J Kirkhorn, S. Frigstad a H. Torp, “Comparison of Pulse Inversion and Second Harmonic for Ultrasound Contrast Imaging”, 2000 IEEE Ultrasonic Symposium Proc., 1897-1901. In the present invention, a particular multi-pulse beamforming technique is used for advantageously shaping the beam pattern of the array.

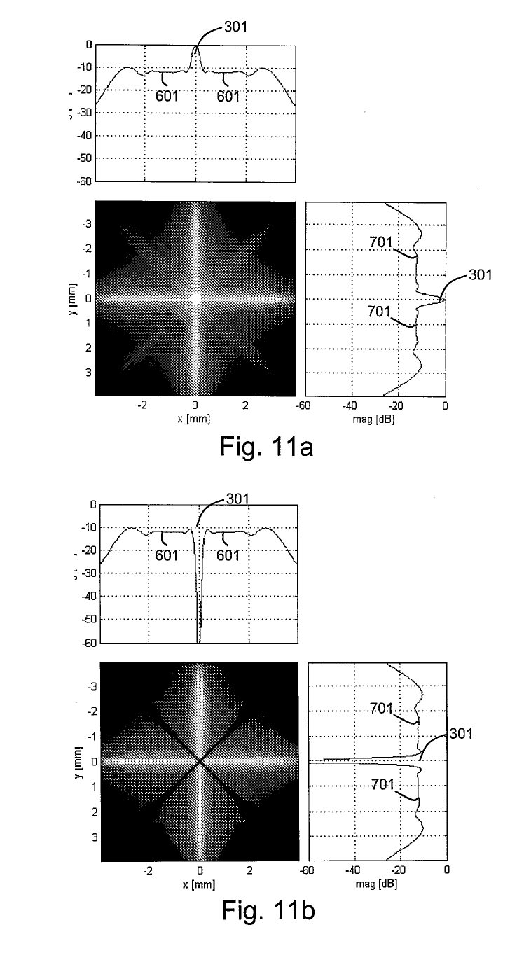

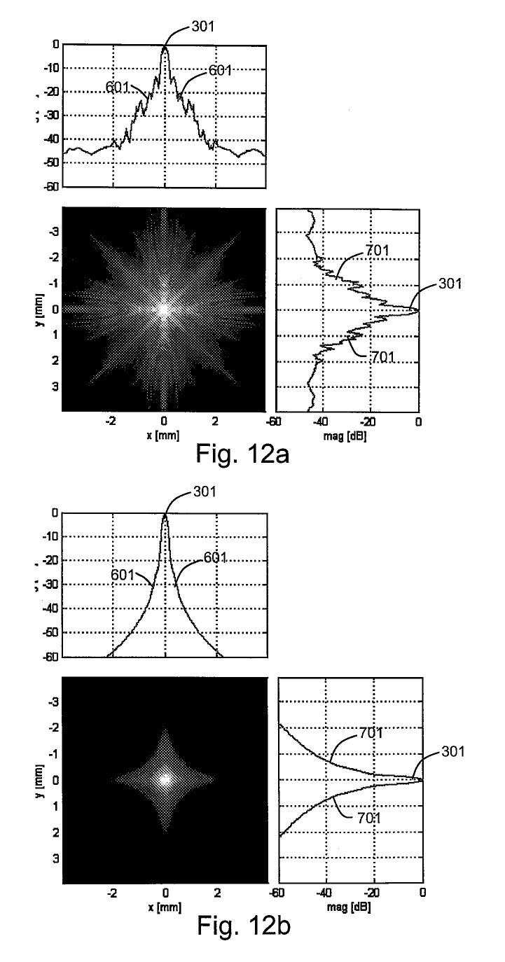

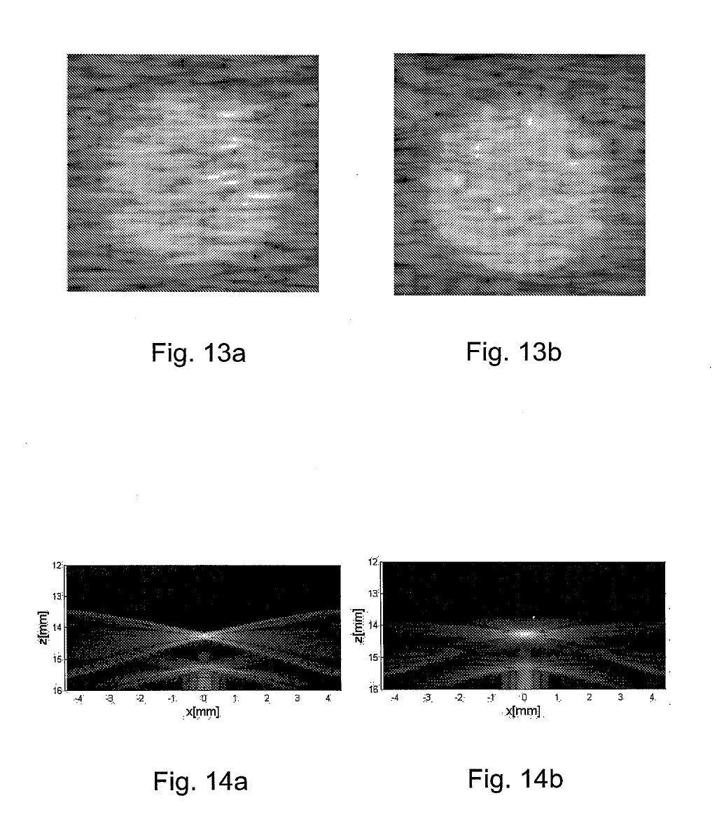

With reference to FIG. 9, during the first pulse firing at instant t1, the X and Y arrays transmit with the same phase through transmission circuits not shown. The received signals Sx(t) and Sy(t) are added in phase thus obtaining the radiation diagram of FIG. 11a in the focal plane. This diagram, as well as the other radiation diagrams shown in the drawings, has been obtained by simulating the behavior of the transducer, in this case a 32+32 element transducer, by means of the software FIELD II, which allows to calculate the acoustic field generated by arbitrary geometry transducers. For details see J. A Jensen, “Simulation of Advanced Ultrasound System Using Field II”, IEEE International Symposium on Biomedical Imaging: Macro to Nano, 2004. As explained above, undesired sidelobes 601, 701 appear along the x and y direction due to the contribution of the main lobes of the two independent X and Y arrays. The sidelobes have a magnitude of −12 db with respect to the central zone of the diagram which represents the main lobe 301 of the transducer. Such sidelobes 601, 701 prevent the transducer from being used in common echographic applications where, as a rough estimate, sidelobes are normally 60 dB less than the main lobe. For this reason a second pulse is transmitted at instant t2, this time with a 180° phase shift between the X and Y array. The received signals Sx(t) and Sy(t) are phase shifted by 180° and added together. The resulting radiation diagram in the focal plane is shown in FIG. 11b. The beam pattern is similar to the one of FIG. 11a, but this time, along the central zone 301 around the acoustic axis of the transducer, the pressure field is almost void. This is due to the fact that the pressure fields generated by the two independent arrays X and Y destructively interfere in the zone of superimposition because of the phase inversion. Thus, if the movement of the tissue can be considered slow enough to allow coherent summation of contribution from different temporal instants, by subtracting the signals S(t) obtained at the instant t1 and t2, the result is a cancellation of the sidelobes. FIG. 12a shows the resulting radiation diagram. The beam pattern is not optimal as the one that can be obtained by a corresponding 2D full-array and which is depicted in FIG. 12b, however sidelobes 601, 701 are strongly reduced and kept to a level which allows to obtain acceptable echographic images as it can be seen in FIG. 13 which shows a comparison between the images obtained on a spherical phantom with a 64-element array according to the invention (FIG. 13a) and a 1024-element full array (FIG. 13b).

From a mathematical point of view, what so far described can be expressed in analytical terms using known models of propagations of ultrasounds such as those disclosed in J. A Jensen, “A model for the propagation and scattering of ultrasound in tissue”, J. Acoustic Soc. Am., 89: 182-191, 1991. Given an acoustic transducer, the signal s(t) received by such transducer can be expressed as

$$s ( t ) = {\int }_{V} v ( t ) {*}_{t} {h}_{t} ( x , y , z , t ) {*}_{x , y , z} f ( x , y , z ) {*}_{t} {h}_{r} ( x , y , z , t ) d x d y d z \qquad ( 3 )$$

where the term hₜ(x,y,z,t) is the spatial impulsive response of the transmitting array, v(t) is the electro-mechanical response of the transducer convolved with the excitation signal, f(x,y,z) represents the tissues to be analysed in terms of variations of density and velocity of the propagation medium which give rise to the scattered filed, hᵣ(x,y,z,t) is the spatial impulsive response of the receiving array and V is the volume of interest.
Equation (3) can be simplified by indicating with

$${h}_{tr} ( x , y , z , t ) = {h}_{t} ( x , y , z , t ) {*}_{x , y , z} {h}_{r} ( x , y , z , t ) \qquad ( 4 )$$

the convolution of the entire system (reception and transmission). It is to be noted that in classic echography where the same transducer is used for transmitting and receiving, the spatial impulsive response of the system is the convolution of the spatial impulsive response of the transducer with itself.
In the specific case of the transducer according to the invention formed by two independent arrays X and Y, s(t) can be expressed, by applying the principle of the superimposition of effects, as a linear combination of 4 terms:

$$\begin{matrix}s ( t ) = {a}_{XX} {s}_{XX} ( t ) + {a}_{XY} {s}_{XY} ( t ) + {a}_{YX} {s}_{YX} ( t ) + {a}_{YY} {s}_{YY} ( t ) = \\ = {\int }_{V} v ( t ) {*}_{t} f ( x , y , z ) {*}_{x , y , z} [ \begin{matrix}\begin{matrix}{a}_{XX} {h}_{XX} ( x , y , z , t ) + \\ \begin{matrix}{a}_{XY} {h}_{XY} ( x , y , z , t ) + \\ {a}_{YX} {h}_{YX} ( x , y , z , t ) +\end{matrix}\end{matrix} \\ {a}_{YY} {h}_{YY} ( x , y , z , t )\end{matrix} ] d x d y d z\end{matrix} \qquad ( 5 )$$

where:
s_{XX}(t) is the signal received from array X when the same array transmits, s_{XY}(t) is the signal received from the array Y when the array X transmits, s_{YX}(t) is the signal received from the array X when the array Y transmits, s_{YY}(t) is the signal received from array Y when the same array transmits;
a_{XX}, a_{XY}, a_{YX} and a_{YY} are coefficients which assume value +1 if the received signal has the same phase of the corresponding transmission signal, the value −1 if the phase is opposite.
   It is to be noted that in (5) it has been hypothesised that the two arrays X and Y have the same electro-mechanic response and are excited with the same pulse (with the exception of the phase).
   No phase inversion is made during the first firing, hence the coefficients a_{XX}, a_{XY}, a_{YX} and a_{YY} are +1. The signal s₁(t) received after the first firing is thus:

$${s}_{1} ( t ) = {\int }_{V} v ( t ) {*}_{t} f ( x , y , z ) {*}_{x , y , z} [ \begin{matrix}\begin{matrix}\begin{matrix}{h}_{XX} ( x , y , z , t ) + \\ {h}_{XY} ( x , y , z , t ) +\end{matrix} \\ {h}_{YX} ( x , y , z , t ) +\end{matrix} \\ {h}_{YY} ( x , y , z , t )\end{matrix} ] d x d y d z \qquad ( 6 )$$

### Being

h_{XY}(x,y,z,t)=h_{YX}(x,y,z,t) (7)

equation (6) can be expressed as:

$${s}_{1} ( t ) = {\int }_{V} v ( t ) {*}_{t} f ( x , y , z ) {*}_{x , y , z} [ \begin{matrix}\begin{matrix}{h}_{XX} ( x , y , z , t ) + \\ 2 {h}_{XY} ( x , y , z , t ) +\end{matrix} \\ {h}_{YY} ( x , y , z , t )\end{matrix} ] d x d y d z \qquad ( 8 )$$

During the second firing, the phase is inverted, hence a_{XX}=a_{YY}=1 and a_{XY}=a_{YX}=−1. The signal s₂(t) received after the second firing is thus:

$${s}_{2} ( t ) = {\int }_{V} v ( t ) {*}_{t} f ( x , y , z ) {*}_{x , y , z} [ \begin{matrix}\begin{matrix}{h}_{XX} ( x , y , z , t ) - \\ 2 {h}_{XY} ( x , y , z , t ) +\end{matrix} \\ {h}_{YY} ( x , y , z , t )\end{matrix} ] d x d y d z \qquad ( 9 )$$

By differentiating at Radio Frequency the two signals s₁(t) and s₂(t), the resulting signal s_{rf}(t) is

$$\begin{matrix}{s}_{rf} ( t ) = {s}_{1} ( t ) - {s}_{2} ( t ) \\ = {\int }_{V} v ( t ) {*}_{t} f ( x , y , z ) {*}_{x , y , z} 4 {h}_{XY} ( x , y , z , t ) d x d y d z\end{matrix} \qquad ( 10 )$$

As it can be seen from equation (10), the spatial impulsive response that can be obtained with the beam-forming above described is equal, apart from a factor 4, to the convolution of the spatial impulsive response of the X array with the spatial impulsive response of the Y array. In other words, the received signal is the same that it would be obtained by transmitting only with the X array and receiving only with the Y array (this modality is hereinafter called “cross”). In classical echography, where the same mono-dimensional array is used both for transmitting and for receiving, the convolution of the spatial impulsive response with itself

$$( {h ( x , y , z , t )}_{t} * h ( x , y , z , t ) )$$

determines a narrow acoustic beam along one of the two directions and a reduction of sidelobes along the same direction as the focusing is made both in transmission and in reception. In the cross modality, instead, the focalization is made with respect to the X-axis only in transmission and with respect to the Y-axis only in reception (or vice versa). Thus the angular resolution that can be obtained with an array in cross modality is, along one of the two directions, less than the resolution that can be obtained with a mono-dimensional array having the same aperture. Furthermore, in cross modality it is not possible to operate a dynamic focusing in reception along both directions x and y. However, if the difference between the two signals s₁(t) and s₂(t) is operated after the detection of the envelope of each signal, the performance of the transducer can be improved (this modality is hereinafter called “interleaved”).

In this case the signal containing the echographic information to be processed is called sₑₙᵥ(t) which is the modulus of the difference between the envelope of the signal received after the first firing and the envelope of the signal received after the second firing:

$$\begin{matrix}{s}_{env} ( t ) = \vert env ( {s}_{1} ( t ) ) - env ( {s}_{2} ( t ) ) \vert \\ = \vert \begin{matrix}env ( {s}_{XX} ( t ) + 2 {s}_{XY} ( t ) + {s}_{YY} ( t ) ) - \\ env ( {s}_{XX} ( t ) - 2 {s}_{XY} ( t ) + {s}_{YY} ( t ) )\end{matrix} \vert \\ = \vert \begin{matrix}env ( {\int }_{V} v ( t ) {*}_{t} f ( x , y , z ) {*}_{x , y , z} [ \begin{matrix}\begin{matrix}{h}_{XX} + \\ 2 {h}_{XY} +\end{matrix} \\ {h}_{YY}\end{matrix} ] d x d y d z ) - \\ env ( {\int }_{V} v ( t ) {*}_{t} f ( x , y , z ) {*}_{x , y , z} [ \begin{matrix}\begin{matrix}{h}_{XX} - \\ 2 {h}_{XY} +\end{matrix} \\ {h}_{YY}\end{matrix} ] d x d y d z )\end{matrix} \vert\end{matrix} \qquad ( 11 )$$

For the sake of readability, equation (11) does not indicate the dependencies of the spatial impulsive responses from the spatial and temporal variables (x,y,z,t).
The presence in (11) of non-linear operators for the detection of the envelope does not allow a simple analytical handling of the math. However, the improvement in the beam formation can be understood by noting that by subtracting the envelope of the signal s₂(t) from the envelope of the signal s₁(t), contrary to what happens by differentiating at RF as seen above, there's no total cancellation of signals s_{XX}(t) and s_{YY}(t) for any value of time t. Particularly, for t˜2z_{f}/c (where c is the speed of sound in the medium and z_{f} the focal distance), the signal received with the second firing is null, while the signal received with the first firing is given by s_{XY}(t) strengthened by the signals s_{XX}(t) and s_{YY}(t) which add in phase, thus increasing the power of the signal in the zone of interest (in the proximity of the focus) with respect to the cross modality where only s_{XY}(t) is received. Furthermore, by differentiating the signals after the envelope detection it is possible to dynamically focus in reception along both the directions X and Y, thus keeping a good resolution in a wider region of the space.

This finding has been tested experimentally by calculating the point spread function (psf) of the cross and interleaved modality. FIG. 14 shows the psf that are obtained by operating the difference of the received signals after the two firings respectively before (FIG. 14a, cross modality) and after (FIG. 14b, interleaved modality) the envelope detection. The images of the figure are obtained by placing a point-like scatterer in front of the array at coordinates (0,0,z_{f}), where z_{f} is the focal distance, and simulating the electronic sweeping of the acoustic beam along the X-axis. In other words, the psf is the B-Mode echographic image of a point-like scatterer. As it can be noted from the comparison of the two psf, in the right image (interleaved modality) the acoustic beam is better focused and the sidelobes are less extended along the X direction with respect to the left image (cross modality).

The new beam-forming technique so far disclosed is substantially based on the idea of generating an optimized acoustic field distribution by exploiting the physical phenomenon of phase cancellation which allows to cancel or at least reduce the undesired components responsible of sidelobes formation. This is achieved by generating an auxiliary field essentially containing only the undesired components, mainly out of axis, which auxiliary field is subtracted from the main field, thus obtaining the desired filtering effect.

This technique has been mainly devised for being used in combination with a transducer according to the present invention, however the skilled person would appreciate that it can also be advantageously used with any kind of transducer arrays, especially when there's the need to optimize the beam formation with a reduced number of echographic channels.

While the preferred embodiment of the invention has been illustrated and described in the drawings and foregoing description, the same is to be considered as illustrative and not restrictive in character, it being understood that all changes and modifications that come within the spirit of the invention are desired to be protected.

1-37. (canceled)

38. An ultrasound transducer comprising an array of electro-acoustic micro-cells (102, 102′, 103, 103′, 104), a first (302, 303, 304) and a second (402, 403, 404) group of transducer elements arranged substantially along two directions (x, y), each element being defined by a group of micro-cells of the array (102, 102′, 103, 103′, 104), at least part of the micro-cells (102, 103, 104) of each group being electrically interconnected by a first connection pattern having shape with main orientation along one of the two directions (x, y) and substantially extending along the entire element, characterized in that each group of micro-cells (102, 102′, 103, 103′, 104) defining each element (302, 303, 304) comprises micro-cells (102′, 103′, 104) interconnected by further connection pattern or patterns having shape with main orientation along the other of the two directions (y, x), such further connection pattern or patterns being electrically isolated from the first connection pattern.

39. The ultrasound transducer according to claim 38, wherein the elements of the first group (302, 303, 304) and the elements of the second group (402, 403, 404) are arranged to form two mono-dimensional arrays (X₁ . . . Xₙ, Y₁ . . . Yₘ) respectively oriented along the x and y direction.

40. The ultrasound transducer according to claim 39, wherein the two mono-dimensional arrays (X₁ . . . Xₙ, Y₁ . . . Yₘ) are at least partially overlapped.

41. The ultrasound transducer according to claim 39, wherein the elements (X₁ . . . Xₙ, Y₁ . . . Yₘ) are formed by connecting micro-cells with metallization patterns provided in at least two layers of the transducer.

42. The ultrasound transducer according to claim 41, wherein the metallization patterns on the first layer have the form of curved and/or polygonal lines substantially defining the direction of the elements of the first group (x) and the metallization patterns on the second layer have the form of curved and/or polygonal lines substantially defining the direction of the elements of the second group (y).

43. The ultrasound transducer according to claim 42, wherein the lines on different layers are crossed, the layers being isolated, the number of lines on the first layer defining the number of elements of the first group (X₁ . . . Xₙ) and the number of lines on the second layer defining the number of elements of the second group (Y₁ . . . Yₘ).

44. The ultrasound transducer according to claim 38, wherein the micro-cells have first (17) and second (18) electrodes, each element comprising a group of micro-cells having the first electrodes (17) connected together, the connection pattern of said first electrodes defining a path substantially along the corresponding direction of the element.

45. The ultrasound transducer according to claim 44, wherein the micro-cells forming the elements of the first group (X₁ . . . Xₙ) are connected on a first layer, while the micro-cells forming the elements of the second group (Y₁ . . . Yₘ) are connected on a second layer.

46. The ultrasound transducer according to claim 45, wherein the second electrode (18) of the micro-cells of the array is commonly connected and each element of a group is formed by micro-cells of one group having first electrodes (17) connected together to define a path substantially along the direction of the element (x, y) and by interleaved micro-cells of other subgroups, the first electrodes (17) of said micro-cells of the other groups being so connected to define a path substantially along the direction of the elements (y, x) of the other group.

47. The ultrasound transducer according to claim 38, wherein each element of the first group (302, 303, 304) comprises intermingled subgroups of micro-cells belonging to elements of the second group (402, 403, 404).

48. The ultrasound transducer according to claim 47, wherein each element of the first group (302, 303, 304) is defined by subgroups of micro-cells connected together alternated by subgroups of micro-cells connected to form elements of the second group (402, 403, 404) in a chess-board-like disposition.

49. The ultrasound transducer according to claim 38, wherein each element of a group (302, 303) is formed by an elementary matrix (202, 203) of interconnected micro-cells having an arbitrary shape repeated along the element to fill a rectangular area of the array, the long axis of the rectangle defining the direction of the element (x).

50. The ultrasound transducer according to claim 49, wherein part of the elementary matrixes (202, 203) of the area are connected together to define a pattern substantially along the direction of the element (x), the remaining elementary matrixes (202′, 203′) of the element being partially connected together according to patterns substantially parallel to the short axis of the rectangle (y).

51. The ultrasound transducer according to claim 50, wherein the patterns substantially parallel to the short axis (y) of the rectangle are oriented along directions parallel to the long axis (x) of the rectangles forming the elements of the other group (402, 403).

52. The ultrasound transducer according to claim 51, wherein the elementary matrixes (103, 103′, 203, 203′) have the shape of quadrilaterals interconnected through the vertexes.

53. The ultrasound transducer according to claim 52, wherein two consecutive quadrilaterals (202, 202′, 203, 203′) are connected only on one vertex of each quadrilateral.

54. The ultrasound transducer according to claim 53, wherein at least one side of the quadrilaterals is parallel to one of the axis of the rectangle.

55. The ultrasound transducer according to claim 53, wherein the sides of the quadrilaterals (203, 203′) are oblique with respect to the axes of the rectangle, the connection path between two vertexes being parallel to one of the axis of the rectangle (x, y).

56. The ultrasound transducer according to claim 38, wherein the micro-cells are arranged in a matrix with rows and columns respectively placed along the horizontal x and vertical y axis, each element (302, 303) of a first mono-dimensional array (X₁ . . . Xₙ) along the x axis being formed by alternatively connecting subgroups of micro-cells with vertical connecting lines having typically a zigzag behavior, each element of a second mono-dimensional array (Y₁ . . . Yₘ) along the y axis being formed by alternatively connecting subgroups of micro-cells with horizontal connecting lines having typically a zigzag behavior in a resulting interlaced matrix arrangement.

57. The ultrasound transducer according to claim 56, wherein each element of a group (304) comprises subgroups of micro-cells having first and second electrodes, the second electrodes (18) connected together, the connection pattern (305, 405, 505) of such second electrodes defining a path substantially along the direction of the elements of another group (404).

58. The ultrasound transducer according to claim 57, wherein each element of a group (304) shares at least part of the micro-cells of elements of said another group (404).

59. The ultrasound transducer according to claim 58, wherein each element (304, 404) is rectangularly shaped, the elements of the two groups being orthogonal, each element of a group (304) being formed by micro-cells (104) also belonging to elements of the other group (404).

60. The ultrasound transducer according to claim 59, wherein any element of a group (304, 404) is formed by micro-cells (104) having first (17) or second (18) electrodes interconnected by at least a metallization pattern substantially extending along the entire surface of the element, the micro-cells forming an element of the first group (304) having the first electrodes (17) interconnected and the micro-cells forming an element of the second group (404) having the second electrodes (18) interconnected or vice versa.

61. The ultrasound transducer according to claim 60, wherein each element of a group (304, 404) is defined by the metallization pattern interconnecting the first electrodes (17) and each element of the other group (404, 304) is defined by the metallization pattern interconnecting the second electrodes (18) or vice versa.

62. The ultrasound transducer according to claim 38, wherein the micro-cells are connected to define a bidimensional array of cross elements arranged in rows and columns.

63. The ultrasound transducer according to claim 62, wherein connecting pads are provided to allow the connection of each row and each column of the array to electronic driving means.

64. The ultrasound transducer according to claim 38, wherein the micro-cells are placed on a curved surface.

65. The ultrasound transducer according to claim 38, characterized by the fact that said ultrasound transducer is provided in combination with an electronic circuit (106, 206, 306) comprising driving means for independently driving the elements of the transducer.

66. The ultrasound transducer according to claim 65, wherein said electronic circuit comprises a first (106) and a second (206) beam-former, the rows of the transducer being connected or connectible to said first beam-former (106), the columns of the transducer being connected or connectible to said second beam-former (206), said first and said second beam-formers (106, 206) comprising amplifying and/or delaying and/or summing elements to achieve an independent focusing of the beams generated/received by the columns and/or the rows of the array.

67. The ultrasound transducer according to claim 66, wherein the two beam-formers (106, 206) are provided for allowing the focusing of the beam received from the transducer, the first beam-former (106) having an input for the connection with the rows and an output for providing a first focused signal (S_{y}(t)), the second beam-former (206) having an input for the connection with the columns of the transducer and an output for providing a second focused signal (Sₓ(t)).

68. The ultrasound transducer according to claim 67, wherein the first (S_{y}(t)) and the second (Sₓ(t) focused signals are combined through a non-linear circuit (306), said non-linear circuit design characteristics being selected from the group consisting of a multiplier circuit, a logarithmic circuit, and a cross-correlation circuit to provide a resulting focused signal (S(t)).

69. The ultrasound transducer according to claim 68, wherein the first (S_{y}(t)) and the second (Sₓ(t)) focused signals are combined after the envelope of each signal has been extracted by one or more envelope detector circuits.

70. The ultrasound transducer according to claim 69, wherein the electronic circuit and the transducer are integrated on the same chip.

71. An ultrasound imaging apparatus comprising a front-end having a number of channels for driving ultrasonic transducers, said channels comprising transmitting and receiving means, characterized by the fact that said ultrasound imaging apparatus is provided in combination with a transducer constructed and arranged according to claim 38.

72. The ultrasound imaging apparatus according to claim 71, wherein at least two beam-formers and control means are provided for controlling the beam formation, said means being configured to perform the following steps:
setting the focusing parameters of each of the two beam-formers;
generating a first transmitting pulse;
feeding each of the two beam-formers with such pulse;
receiving the echo;
achieving an independent beam-forming in reception with each of the two beam-formers;
adding the resulting focused signals to obtain a signal S₁(t);
generating a second transmitting pulse;
feeding one of the two beam-formers with such pulse;
feeding the other of the two beam-formers with the same pulse 180°-phase-shifted;
receiving the echo;
achieving an independent beam-forming in reception with each of the two beam-formers;
adding the resulting focused signals after a 180°-phase shift to obtain a signal S₂(t);
subtracting the S₁(t) and S₂(t) signals to obtain the signal S(t);
extracting the envelope of signal S(t) to be used for imaging purpose.

73. The ultrasound imaging apparatus according to claim 72, modified in that the signal S(t) to be used for imaging purpose is obtained by subtracting the envelope of the S₁(t) and S₂(t) signals.

74. A method for achieving an optimized beam formation with a transducer having superimposed elements (x, y) according to claim 38, said method comprising the following steps:
transmitting a first pulse by the elements causing a target to generate a first echo signal;
transmitting a second pulse by the elements causing the target to generate a second echo signal; and
reversing the phase of the pulse transmitted by the x and y elements during the second transmission so that a sidelobe reduction of the resulting beam can be obtained when the echo signals are combined.
